(19) United States
(12) Patent Application Publication
(10) Pub. No.: US 2008/0051773 A1
(43) Pub. Date: Feb. 28, 2008

(54) Automated Treatment of Psoriasis

(76) Inventors: Sergei Ivanov, Newton, MA (US); Robert H. Webb, Lincoln, MA (US); Richard Rox Anderson, Boston, MA (US)

Correspondence Address: FISH & RICHARDSON PC, P.O. BOX 1022, MINNEAPOLIS, MN, 55440-1022, US

(21) Appl. No.: 11/758,067

(22) Filed: Jun. 5, 2007

Related U.S. Application Data
(63) Continuation-in-part of application No. 11/293,905, filed on Dec. 5, 2005.
Application No. 11/293,905 is a continuation of application No. 10/224,059, filed on Aug. 20, 2002, now Pat. No. 6,984,228.
Application No. 10/224,059 is a continuation of application No. 09/169,083, filed on Oct. 8, 1998, now Pat. No. 6,436,127.
(60) Provisional application No. 60/061,487, filed on Oct. 8, 1997.
(60) Provisional application No. 60/811,309, filed on Jun. 5, 2006.

(51) Int. Cl. A61N 5/067

(52) U.S. Cl. 606/12; 600/407; 607/89

(57) ABSTRACT

Treating a skin condition with electromagnetic radiation includes receiving the radiation at an image-shaping device, and causing the image-shaping device to form a shaped treatment image including the electromagnetic radiation on a patient's skin based on the skin condition.

### CLAIM OF PRIORITY

This application is a continuation in part of and claims priority under 35 U.S.C. 120 to U.S. patent application Ser. No. 11/293,905, filed on Dec. 5, 2005, which is a continuation of and claims the benefit of U.S. patent application Ser. No. 10/224,059, filed Aug. 20, 2002, and issued as U.S. Pat. No. 6,984,228, which is a continuation of and claims the benefit of U.S. patent application Ser. No. 09/169,083, filed on Oct. 8, 1998 and issued as U.S. Pat. No. 6,436,127, which claims priority to Provisional Application No. 60/061,487, filed on Oct. 8, 1997, the contents of which are incorporated herein by reference in their entirety

This application also claims priority under 35 U.S.C. 119(e) to Provisional Patent Application No. 60/811,309, filed Jun. 5, 2006, the entire contents of which are hereby incorporated by reference.

### TECHNICAL FIELD

This disclosure relates to automated treatment of psoriasis.

### BACKGROUND

Millions of people around the world have psoriasis, a chronic disease characterized by sharply demarcated erythematous plaque with a silvery white scale. Psoriasis effects 3 to 5 million Americans and up to 3 percent of populations worldwide. Most common sites of involvement are scalp, elbows and knees, followed by nails, hands, feet and trunk. Psoriatic arthritis also occurs in 5-30% of the patients with cutaneous psoriasis. Most patients with psoriasis tend to have the disease for life. While psoriasis is not typically life threatening, it can greatly affect appearance, self-esteem and overall quality of life.

Numerous topical and systemic therapies are available for the treatment of psoriasis. Treatment modalities are chosen on the basis of disease severity, patient preference and response. One treatment option for patients with moderate to severe psoriasis is the use of various forms of phototherapy. Exposure to sunlight is perhaps the oldest treatment form for psoriasis. Other developed options include broad-band ultraviolet B (UVB) radiation and narrow band UVB, photochemotherapy with psoralen plus ultraviolet A (PUVA), and UV laser. The short term side effects of phototherapy include burning, while long term side effects may include premature aging and increased risk of skin cancer.

The use of UVB can be effective in both thin plaque disease and thick plaque disease. The delivery of UVB therapy can provide effective treatment for patients without the need for systemic agents. The maturation and development of various forms of UVB treatment have led to the use of more precise wavelengths within the UVB range that have the most effective therapeutic benefit for psoriasis. Most equipment in the U.S., however, emits broad-band UVB light. Newer equipment, which produces narrow-band light, results in faster improvements with longer remission.

Laser emitting in UVB wavelengths within the action spectrum of psoriasis have enhanced the range of phototherapy devices utilized. One example of the enhancement of the phototherapy treatment of psoriasis has been the use of the 308 nm excimer laser, which is starting to have a more prominent role in psoriasis treatment. The laser allows treatment on only the involved skin, thus considerably higher doses of UVB can be administered to the psoriatic plaque at a given treatment compared with traditional phototherapy.

### SUMMARY

In general, in one aspect, disclosed is an apparatus for treating a skin condition. The apparatus includes a source of electromagnetic radiation, an image-shaping device configured to receive the electromagnetic radiation from the source, and a control system configured to cause the image-shaping device to form a shaped treatment image including the electromagnetic radiation on a patient's skin based on an image of the skin condition.

Implementations of the apparatus may include one or more of the following features.

The image-shaping device can reflect or transmit the electromagnetic radiation to direct it towards the patient.

The source of electromagnetic radiation can include a laser or an arc-lamp. The apparatus can include optics to couple the radiation from the source to the image-shaping device. Furthermore, the apparatus can include an optical system to couple the electromagnetic radiation from the image-shaping device to a patient's skin. For example, the optical system can include an objective lens configured to focus electromagnetic radiation from the image-shaping device onto a patient's skin.

The apparatus can also include an image-capture device (for example, a color video camera). For example, the control system can be configured to identify the skin condition in an image acquired from the image-capture device to cause the image-shaping device to form the shaped treatment image on the patient's skin. In one example, the control system is configured to identify the skin condition by recognizing a psoriatic plaque in the image acquired from the image-capture device.

In some implementations, the control system is configured to identify the skin condition by dividing the image acquired from the image-capture device into components having at least red and green values, and comparing the red and green values. In some cases, the control system is also configured to normalize the red and green values. Moreover, the control system can be further configured to assign a value to each pixel in the mask image based on the comparison between the red and green values. Furthermore, the control system can also be configured to modify the mask image by changing the value of the pixel for each pixel in the mask image, if a threshold number of surrounding pixels have a different value.

The control system can be configured to cause the image-shaping device to form the treatment image by generating a mask image corresponding to the skin condition, communicating the mask image to the image-shaping device, causing the image-shaping device to form an image corresponding to the mask image, and causing an optical system to focus light from the image-shaping device onto the patient's skin in such a way that the image corresponding to the mask illuminates the skin condition and does not illuminate skin not bearing the condition.

The image-shaping device can include a plurality of pixels, and the control system can be configured to cause the image-shaping device to divide the electromagnetic radiation from the source into one beam for each pixel, and to cause a subset of the pixels to direct corresponding beams onto the patient's skin. The control system can also be configured to repeatedly identify the skin condition in an updated image acquired from the image-capture device, and cause the image-shaping device to update the treatment image based on the skin condition identified in the updated image, and in which the control system is configured to acquire the updated image and update the treatment image at a rate of at least once per second.

The image-shaping device can include a digital micromirror device, a liquid crystal on silicon device, or a liquid crystal display.

The electromagnetic radiation can include ultraviolet radiation, for example, electromagnetic radiation at a wavelength of around 308 nm.

In certain implementations, the image-shaping device is configured to deliver electromagnetic radiation having an intensity of around 1 to 100 mJ/cm² at the patient's skin. The image-shaping device is configured to project the treatment image onto an area of skin of around 4 to 400 cm².

The image-shaping device can be configured to receive a beam of electromagnetic radiation from the source and to simultaneously project a plurality of beams derived from the received beam to form the treatment image.

In another aspect, a method is disclosed for treating a skin condition with electromagnetic radiation. The method includes receiving the radiation at an image-shaping device, and causing the image-shaping device to form a shaped treatment image including the electromagnetic radiation on a patient's skin based on the skin condition.

Embodiments of the method may further include any of the features corresponding to those set forth above for the apparatus aspect.

In yet another aspect, disclosed is a method for treating a skin condition that includes receiving an image of a skin condition, projecting a treatment image based on the received image onto a patient's skin bearing the skin condition, and repeatedly, at a rate of at least once per second, receiving an updated image of the skin condition, and updating the projected treatment image based on the updated received image.

Embodiments of the method may further include any of the features corresponding to those set forth above for the apparatus aspect.

In yet another aspect, disclosed is a method for treating a skin condition with electromagnetic radiation that includes receiving an image of a skin condition, and simultaneously projecting a plurality of beams of the electromagnetic radiation onto a patient's skin in a pattern based on the received image.

Embodiments of the method may further include any of the features corresponding to those set forth above for the apparatus aspect.

The details of one or more embodiments of the invention are set forth in the accompanying drawings and the description below. Other features will be apparent from the description and the claims.

### DESCRIPTION OF DRAWINGS

FIG. 1 is a diagram of an optical treatment system.

FIGS. 2A and 2B are flow charts.

FIG. 3 is a side view of an optical treatment system.

Like reference symbols in the various drawings indicate like elements.

### DETAILED DESCRIPTION

In some examples, to treat psoriatic plaques and other skin conditions, a shaped illumination pattern can be formed with numerous beams of electromagnetic radiation, e.g., laser beams, to fill in a pattern corresponding to the plaque. A map of where to form the pattern and expose the skin to the radiation and where to not expose it is created by a machine-vision system. To allow for fast updates between diagnostic image acquisition and projection of the treatment image, an imaging-shaping device is used that can divide a laser into numerous beams and simultaneously project them over a broad region of skin. This results in numerous beams of reduced intensity, allowing more thorough coverage with less chance of damage to unaffected areas than, for example, scanning a more-intense single beam over the skin. It also allows the image to be updated in real-time, compensating for patient movement during the treatment.

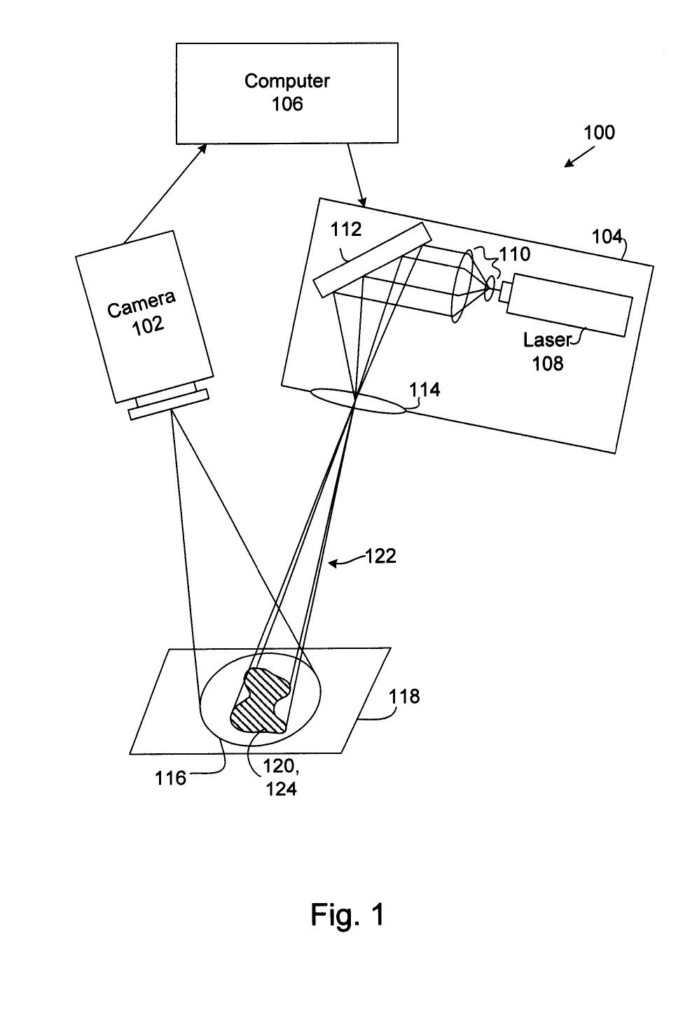

In some examples, as shown in FIG. 1, a treatment system 100 includes a camera 102 and a projection system 104 linked by a computer 106. The projection system 104 includes a UV laser 108, an optical system 110, an image-shaping device 112, and a focusing objective 114 (e.g., one or more lenses). The UV laser 108 could be replaced by other light sources, such as an arc lamp and appropriate filters. The image-shaping device 112 could be a digital micro-mirror device (DMD), a liquid-crystal on silicon (LCoS) device, or another image-shaping device that can divide a light source into numerous individual beams of light and control whether to reflect or transmit each of them individually. The camera 102 is used to capture images of an area 116 of the patient's skin 118. The computer 106 identifies psoriatic plaques 120 or other skin conditions to treat, and generates and transmits an appropriate treatment image to the projecting system 104. Ultraviolet light 122 from the laser 108 is focused by the optical system 110 onto the image-shaping device 112. The image-shaping device 112 generates the image from the computer 106 and imparts that image into the light 112 as it reflects it onto the patient's skin 118, through the focusing lens 114, to form a treatment image 124. In other examples, the image-shaping device can be a transmissive device, so that it directs the light 112 to the patient by transmitting some or all of the light toward the patient (e.g., by selective attenuation, refraction, and/or diffraction). As shown in FIG. 1, the treatment image 124 matches the plaque 120, so that only the plaque 120 is exposed to the UV light 122. The computer 106 continually updates the treatment image 124 generated by the projecting system 104 to match updated images from the camera 102 to compensate for movement of the patient.

To allow accurate identification of the skin conditions to be treated, the camera 108 is selected to have a sufficient resolution and accuracy. The camera is also selected to have a sufficient refresh rate to allow the treatment image to be updated quickly enough to compensate for patient movement. One source of movement of a patient's skin is breathing, which generally has a frequency of about 0.2 Hz. A refresh rate of 1 Hz would provide five updates per breath, which should be sufficient to compensate for any patient movement due to breathing. Faster refresh rates may be needed to compensate for voluntary movement.

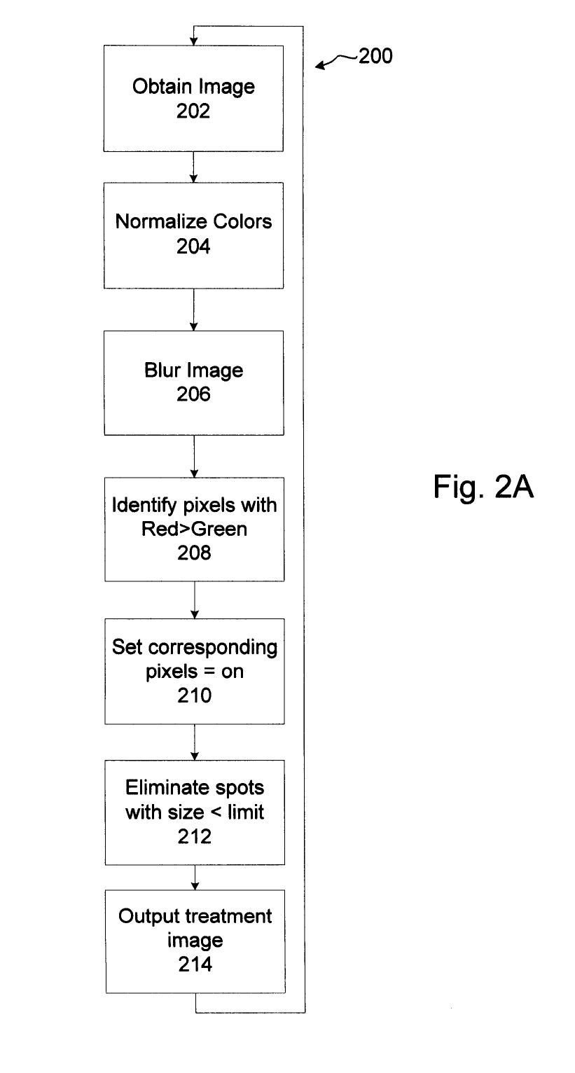

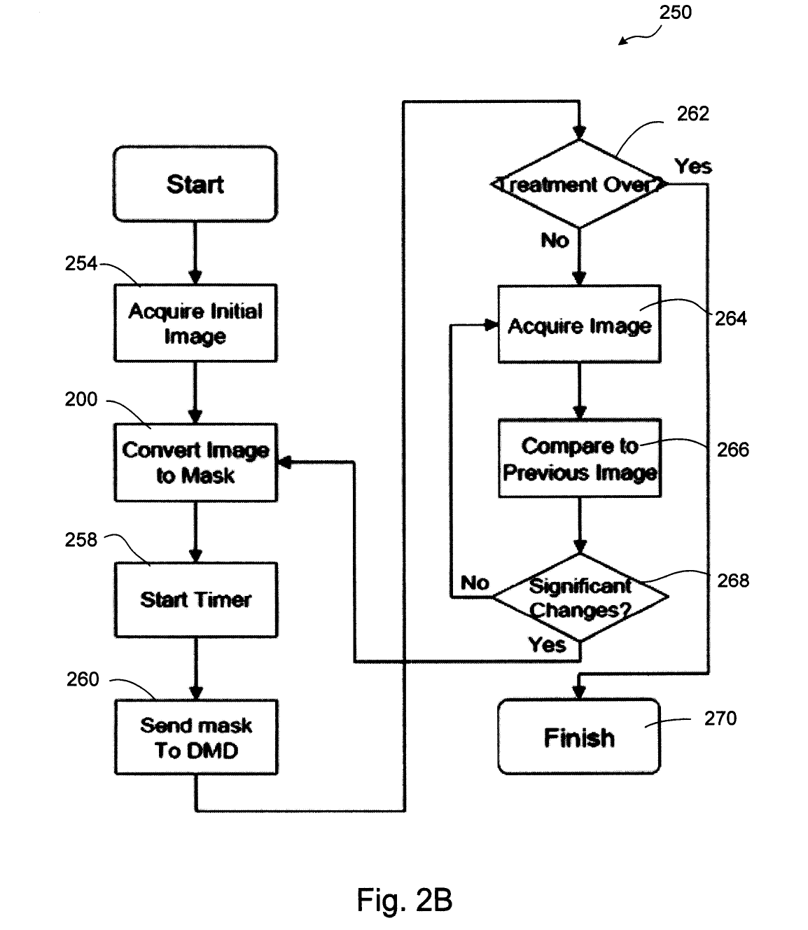

To recognize psoriatic plaques 120 or other skin conditions, the computer 106 employs a machine vision system. In some examples, the machine vision system uses the process 200 shown in FIG. 2A. A color image (e.g., red, green, and blue color planes) is obtained from the camera 102 (step 202). The red and green color planes of the image are normalized by setting the pixels of highest intensity of each color to a value of 255 while setting the pixels with the lowest intensity for each color to a value of 0 (step 204). The blue color plane may also be normalized, for example, to simplify use of standard color processing tools and to allow more detailed pattern-recognition algorithms. Next, the image is blurred using a low-pass spatial frequency filter (step 206). To generate the treatment image, pixels are identified as exhibiting plaque (step 208) based on whether the red to green ratio exceeds one or more threshold values, and corresponding pixels in the treatment image are set to receive illumination (step 210). Increased reflectance in red wavelengths relative to green may indicate an increased blood content, an indicator of erythema. The treatment image may be a monochrome image mask, with white pixels receiving illumination and black pixels blocking the UV light. In some examples, the machine vision algorithm may indicate a dosage for individual areas and the treatment image may be a grayscale image, with different brightness levels indicating different amounts of exposure to UV light. After generating the initial treatment image, spots smaller than a predefined size are eliminated by changing them to match the surrounding pixels (step 212). The resulting treatment image is output to the imaging system 104 (step 214) and the process repeats. To recognize movement of the patent, standard techniques such as dynamic edge detection can also be used. In some examples, other machine vision algorithms are used to recognize areas of the skin needing treatment and to produce the treatment image.

Once the treatment image is generated by the machine vision process, it is projected by the imaging system 104 according to the process 250 in FIG. 2B. First, an initial image is acquired (step 254) and sent to the process 200 where the treatment image mask is generated. When the treatment image is ready, a timer is started for recording and setting the desired exposure time (step 258) and the mask data is sent to the image-shaping device (step 260). While the process 250 is ongoing, light from the light source 108 is shaped by the imaging-shaping device 112 and the treatment image is projected onto the patient. If the exposure time has not yet passed (step 262), a new image is acquired (step 264) and compared to the previous image (step 266). If significant changes are detected (step 268), the updated image is sent to the process 200 to have a new treatment image mask generated. Depending on the degree to which the target area is changes, the timer (step 258) may or may not be reset. For example, if the changes are small so that substantially the same region is being exposed, the timer is not reset as there is no need to adjust the overall exposure time. The new mask is sent to the image-shaping device to update the projected image (step 260). Once the programmed exposure time has passed (step 262), the process 250 is finished (step 270) and the light source 108 is turned off.

In some examples, a 308 nm excimer laser is used as the light source 108. This frequency has therapeutic value in treating psoriatic plaques. Other sources of illumination at the same or other frequencies may be used, depending on the skin condition being treated and the energy levels required. In the case of a laser, the optical system 110 typically includes a diverging lens 110a to expand the laser beam to a radius sufficient to illuminate the image-shaping device 112 and a collimating lens 110b to re-collimate the light at that radius. After the image-shaping device 112 imparts the treatment image to the collimated light, it is focused by the lens 114 onto the patient's skin. In some examples, the lens 114 is selected to have a 50 cm focal depth, as is common in commercial projectors for visible-light applications, such as television or computer projectors. In some examples, the lenses are made of quartz and are achromatic so that they will not affect the frequency of the light. In some examples, the lenses do not need to be achromatic. Other materials may be used depending on the frequency of light 122 produced by the laser 108 or other light source and the frequencies needed for treatment. In other embodiments, the optical system may be based on reflective optics or a combination of reflective and refractive optics.

To project the expanded and collimated laser beam 122 onto the patient's skin 118 and form the treatment image 124, the image-shaping device 112 reproduces the treatment image determined by the computer 106. In some examples, a Digital micro mirror device (DMD) manufactured by Texas Instruments can be used as the image-shaping device. Such DMDs typically have an array of 16-μm-square mirrors separated by 1 μm gaps. Each mirror is individually controllable to reflect or divert incident light. Each mirror is mounted on a yoke and hinge, which in turn are mounted on a hinge pedestal. A landing electrode and a bias electrode control the position of the mirror, reflecting light into or away from the image area. A spring tip returns the mirror to its neutral position when no force is applied by the electrodes and. The array of mirrors are mounted over an SiO₂ insulation layer over CMOS circuitry and a substrate.

Each mirror corresponds to one pixel of the treatment image. The mirrors are positioned individually or in groups, and once positioned, may remain in position until reset. When the expanded and collimated laser beam 122 is reflected by these mirrors, the result is up to 700,000 or more parallel or slightly diverging beams simultaneously projected to form the treatment image when they reach the patient's skin 118. The use of a dynamic image-shaping device such as a DMD provides numerous options in the design of the imaging system. In some examples, the mirrors can be angled as a set with a net curvature such that the resulting beams are focused as if the mirror array were a single concave or convex mirror, eliminating the need for focusing lens 114. In other examples, different optical systems 110 and/or focusing lenses 114 can be used in combination with one or more active image-shaping devices 112, providing a range of options in the design of a treatment system.

Another technology that can provide numerous controllable beams from a single collimated source is liquid crystal on silicon (LCoS). An LCoS device includes a liquid crystal display integrated with a reflective surface. Each pixel of the display acts as a shutter for the portion of the reflective surface below it, such that numerous beams can be reflected by setting the liquid crystal to be transparent at each position where a treatment beam is desired. LCoS has the advantage of eliminating gaps between pixels, allowing for more uniform treatment within the exposed area. Other projection technologies include transmissive LCD displays.

The projected image can be referred to as a virtual mask, because it serves to expose certain areas to the UV radiation 122 while blocking, or masking, other areas, similar to the way a mask is used in photolithography. Each pixel of the image-shaping device is turned on or off to form the desired image, and the image is maintained for the duration of the treatment period, updated to account for patient movement. In some examples, as discussed below, individual pixels can be flashed on and off to provide less than a full dose of UV radiation to the corresponding area of skin, if that is needed for proper treatment.

In some cases, psoriatic plaques and other skin conditions tend to fluoresce in the presence of UV radiation, making it possible to see visually where the UV radiation is striking the skin. This can be used to refine the vision system, by confirming that the treatment image matches the affected area. In some examples, the projected image is occasionally enlarged, and the computer examines the captured image to see where fluorescence is visible. This can be used to supplement or correct the identification of the skin condition made using the method discussed above. The fast response time of the DMD and other image-shaping devices allow such an exposure to be made sufficiently quickly that unaffected skin will not be burned by the UV radiation.

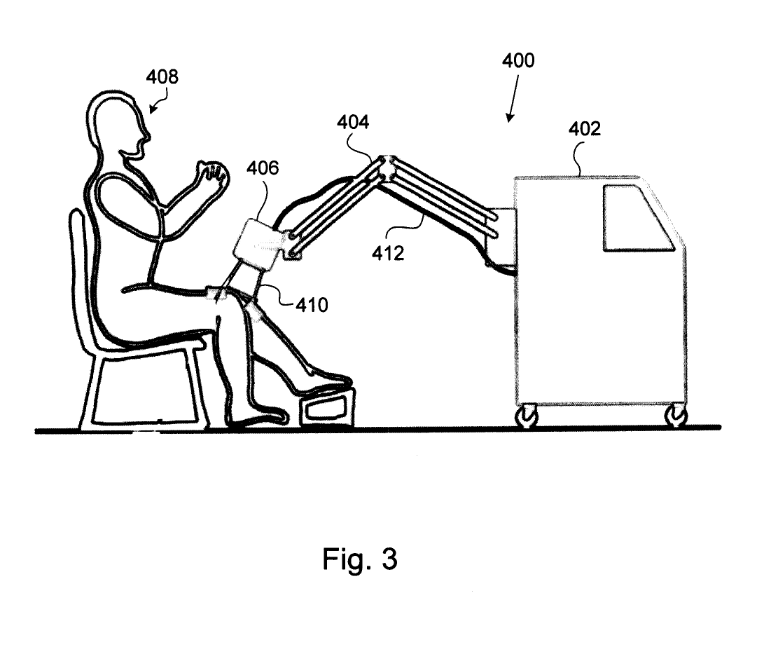

In some examples, the system is integrated into a treatment device as shown in FIG. 3. A treatment device 400 includes a base 402 for housing the laser or other UV light source and control electronics (not shown). A movable arm 404 allows an imaging head 406 to be positioned near the patient 408. An interface fixture 41 0 makes contact with the patient 408's body to keep the imaging head 406 in the proper location. A fiber optic light guide 412 transfers the UV radiation from the light source in the base 402 to the imaging head 406 where the image-shaping device and optics (not shown) are located. The image-shaping device and optics are used to generate the treatment image and project it onto the patient 408's skin. In some examples, the system is fully automated, capable of moving the arm 404 to different areas of the patient's body as required, while in some examples it is positioned manually. The base 402 may include a user interface that attending physicians or nurses may use to control the system, or it may be networked to provide a user interface at a remote control station.

Projecting the treatment image using numerous beams has several advantages. It allows for precise positioning of a therapeutic treatment image to expose a psoriatic plaque or other skin condition to radiation while preventing unaffected skin from exposure. Numerous beams transmitted together, rather than a single beam scanned over the entire treatment area, allow the entire plaque (or at least the portion of the plaque within the treatment area) to be treated at once. The divided beam applies a lower dose, so it can be projected onto an individual area for a longer period of time. With numerous beams projected at once, the total treatment time will be similar to that required to scan a more-intense beam over the same area, with a short exposure time at each point, but with less chance of burning if the beam does reach an unaffected area. It also allows treatment to be more uniform over the affected area.

The lower dose also helps reduce the danger from patient movement, as any exposure of unaffected skin that occurs before the image is updated will have less-severe consequences than it would with an intense beam. In prior systems, a 200 mJ/cm² laser beam would illuminate an area of about 2 cm² for 10 seconds. With an expanded image area of 400 cm², the same laser is reduced in intensity to 100 mJ/cm², and would require around half an our to treat the full imaged area, exposing the entire area (where needed) at once. This approach also avoids the risk of missing or over-treating individual spots that arises in applications with hand-held laser projectors. The dose can be further controlled by rapidly switching the pixels on and off in a region where a lower dose is required. Such modulation is commonly used in commercial DMD and LCoS projectors to illuminate pixels at less-than-full brightness, i.e., shades of grey.

In at least some examples, the system is configured to illuminate a region of the patient's skin with an area of about 4 to 400 cm² with treatment radiation having an intensity of about 100 microJoules/cm² to 1 milliJoule/cm² with an exposure time from about a few tens of second to about one half hour.

The fast image-acquisition rates of digital cameras and fast image-update rates of DMDs also allow the treatment image to be updated in real time to compensate for patient movement. Masking with a digital image-shaping device is easier, cleaner, and faster than masking the skin with sunscreen or manually manipulating the laser to expose only the affected area. The system described can be modified to recognize and treat other skin conditions, such as mycosis fungoides, eczema, actinic keratosis, and lichen planus.

A number of embodiments of the invention have been described. Nevertheless, it will be understood that various modifications may be made without departing from the spirit and scope of the invention. For example, lasers or other light sources producing radiation at other frequencies as appropriate for other skin conditions can be used. Accordingly, other embodiments are within the scope of the following claims.

What is claimed is:

1. An apparatus for treating a skin condition with electromagnetic radiation comprising:
a source of the electromagnetic radiation;
an image-shaping device configured to receive the electromagnetic radiation from the source; and
a control system configured to cause the image-shaping device to form a shaped treatment image comprising the electromagnetic radiation on a patient's skin based on an image of the skin condition.

2. The apparatus of claim 1, in which the image-shaping device reflects the electromagnetic radiation to direct it towards the patient.

3. The apparatus of claim 1, in which the image shaping device transmits the electromagnetic radiation to direct it towards the patient.

4. The apparatus of claim 1 in which the source of electromagnetic radiation comprises a laser.

5. The apparatus of claim 4 also comprising optics to couple a beam from the laser to the image-shaping device.

6. The apparatus of claim 1, in which the source of the electromagnetic radiation comprises an arc-lamp.

7. The apparatus of claim 1 also comprising an optical system configured to couple the electromagnetic radiation from the image-shaping device to a patient's skin.

8. The apparatus of claim 7 in which the optical system comprises an objective lens configured to focus electromagnetic radiation from the image-shaping device onto a patient's skin.

9. The apparatus of claim 1 also comprising an image-capture device.

10. The apparatus of claim 9 in which the control system is also configured to identify the skin condition in an image acquired from the image-capture device to cause the image-shaping device to form the shaped treatment image on the patient's skin.

11. The apparatus of claim 10 in which the control system is configured to identify the skin condition by recognizing a psoriatic plaque in the image acquired from the image-capture device.

12. The apparatus of claim 10 in which the control system is configured to identify the skin condition by
dividing the image acquired from the image-capture device into components having at least red and green values, and
comparing the red and green values.

13. The apparatus of claim 12, in which the control system is also configured to normalize the red and green values.

14. The apparatus of claim 12, in which the control system is also configured to assign a value to each pixel in the mask image based on the comparison between the red and green values.

15. The apparatus of claim 12 in which the control system is also configured to modify the mask image by
for each pixel in the mask image, if a threshold number of surrounding pixels have a different value, changing the value of the pixel.

16. The apparatus of claim 10 in which the control system is configured to cause the image-shaping device to form the treatment image by
generating a mask image corresponding to the skin condition,
communicating the mask image to the image-shaping device,
causing the image-shaping device to form an image corresponding to the mask image, and
causing an optical system to focus light from the image-shaping device onto the patient's skin in such a way that the image corresponding to the mask illuminates the skin condition and does not illuminate skin not bearing the condition.

17. The apparatus of claim 10 in which
the image-shaping device comprises a plurality of pixels, and
the control system is configured to cause the image-shaping device to
divide the electromagnetic radiation from the source into one beam for each pixel, and to
cause a subset of the pixels to direct corresponding beams onto the patient's skin.

18. The apparatus of claim 10 in which the control system is also configured to repeatedly
identify the skin condition in an updated image acquired from the image-capture device, and
cause the image-shaping device to update the treatment image based on the skin condition identified in the updated image,
and in which the control system is configured to acquire the updated image and update the treatment image at a rate of at least once per second.

19. The apparatus of claim 1 in which the image-shaping device comprises a digital micromirror device.

20. The apparatus of claim 1 in which the image-shaping device comprises a liquid crystal on silicon device.

21. The apparatus of claim 1 in which the image-shaping device comprises a liquid crystal display.

22. The apparatus of claim 1 in which the electromagnetic radiation comprises ultraviolet radiation.

23. The apparatus of claim 1 in which the electromagnetic radiation has a wavelength of around 308 nm.

24. The apparatus of claim 1 in which the image-shaping device is configured to deliver electromagnetic radiation having an intensity of around 1 to 100 mJ/cm² at the patient's skin.

25. The apparatus of claim 1 in which the image-shaping device is configured to project the treatment image onto an area of skin of around 4 to 400 cm².

26. The apparatus of claim 1 in which the image-shaping device is configured to receive a beam of electromagnetic radiation from the source and to simultaneously project a plurality of beams derived from the received beam to form the treatment image.

27. The apparatus of claim 9, in which the image-capture device is a color video camera.

28. A method of treating a skin condition with electromagnetic radiation comprising:
receiving the radiation at an image-shaping device; and
causing the image-shaping device to form a shaped treatment image comprising the electromagnetic radiation on a patient's skin based on the skin condition.

29. The method of claim 28 also comprising receiving an image of a skin condition.

30. The method of claim 28 in which receiving the image comprises
receiving an image of the patient's skin from an image capture device, and
identifying regions of the patient's skin having the skin condition to cause the image-shaping device to form the shaped treatment image on the patient's skin.

31. The method of claim 30 in which identifying regions of the patient's skin having the skin condition comprises
dividing the image into components having at least red and green values,
normalizing each component, and
comparing the red and green values.

32. The method of claim 31 in which identifying regions of the patient's skin having the skin condition also comprises normalizing the red and green values.

33. The method of claim 31 in which identifying regions of the patient's skin having the skin condition also comprises assigning a value to each pixel in the mask image based on the comparison between the red and green values.

34. The method of claim 33 also comprising, for each pixel in the treatment image, if a threshold number of surrounding pixels have a different value, changing the value of the pixel.

35. The method of claim 28 in which directing the electromagnetic radiation comprises simultaneously projecting a plurality of beams of the electromagnetic radiation onto the patient's skin in a pattern corresponding to the treatment image.

36. The method of claim 35 in which
generating the electromagnetic radiation comprises energizing a laser,
the image-shaping device comprises a plurality of pixels, and
projecting the plurality of beams comprises
diverging a beam of the electromagnetic radiation in an optical system,
directing the diverged beam onto the image-shaping device,
causing the image-shaping device to divide the diverged beam into one beam for each pixel, and
causing a subset of the pixels of the image-shaping device to direct beams onto the patient's skin.

37. The method of claim 28 also comprising repeatedly, at a rate of at least once per second,
receiving an updated image of the skin condition, and
updating the treatment image formed on the patient's skin based on the updated image of the skin condition.

38. The method of claim 28 in which the image-shaping device reflects the electromagnetic radiation to direct it towards the patient.

39. The method of claim 28 in which the image-shaping device transmits the electromagnetic radiation to direct it towards the patient.

40. The method of claim 28 in which the image-shaping device comprises a digital micromirror device.

41. The method of claim 28 in which the image-shaping device comprises a liquid crystal on silicon device.

42. The method of claim 28 in which the image-shaping device comprises a liquid crystal display.

43. The method of claim 28 in which the skin condition comprises a psoriatic plaque.

44. The method of claim 28 in which the radiation is generated by a laser.

45. The method of claim 44 also comprising using optics to couple a beam from the laser to the image-shaping device.

46. The method of claim 28 in which the radiation is generated by an arc-lamp.

47. The method of claim 28 in which the electromagnetic radiation comprises ultraviolet radiation.

48. The method of claim 28 in which the electromagnetic radiation has a wavelength of around 308 nm.

49. The method of claim 28 in which the electromagnetic radiation has an intensity of around 1 to 100 mJ/cm² at the patient's skin.

50. The method of claim 28 in which the treatment image covers an area of skin of around 4 to 400 cm².

51. The method of claim 28 also comprising
positioning the image-shaping device in proximity to a patient's skin.

52. The method of claim 30 in which the image capture device is a color video camera.

53. A method of treating a skin condition comprising:
receiving an image of a skin condition,
projecting a treatment image based on the received image onto a patient's skin bearing the skin condition, and
repeatedly, at a rate of at least once per second,
receiving an updated image of the skin condition, and
updating the projected treatment image based on the updated received image.

54. A method of treating a skin condition with electromagnetic radiation comprising:
receiving an image of a skin condition, and
simultaneously projecting a plurality of beams of the electromagnetic radiation onto a patient's skin in a pattern based on the received image.
